(19) United States
(12) Patent Application Publication
(10) Pub. No.: US 2012/0290017 A1
(43) Pub. Date: Nov. 15, 2012

(54) Bone fracture fixation clamp

(76) Inventor: George J. Haidukewych, Orlando, FL (US)

(21) Appl. No.: 13/068,397

(22) Filed: May 10, 2011

(51) Int. Cl. A61B 17/84

(52) U.S. Cl. 606/324

(57) ABSTRACT

A fixation clamp to stabilize a bone fracture comprising a pair of arcuate clamp members each including a concave inner surface having at least one spike-like element or protrusion extending outwardly from each concave inner surface to engage the bone surface to secure the fixation clamp in place pivotally coupled to each other and an adjustment device to tighten the arcuate clamp members around the bone forcing the spike-like elements or protrusions into the surface of the bone.

### BACKGROUND OF THE INVENTION

1. Field of the Invention

A fixation clamp to stabilize a bone fracture.

2. Description of the Prior Art

Bone fracture fixation devices are well known in the art. Surgical cables or clamps and metal compression plates are employed in combination with or without fixation screws are common. Cables or clamps and plates are often used alone when the use of screws and plates is not suitable. For example, these devices are frequently used in fixation of long oblique fractures with metal prosthesis that preclude the use of screws for fixation in segments of the bone. Thus the use of cables, wires or clamps are required in such cases without screws. A number of prior art examples are discussed below.

U.S. Pat. No. 4,263,904 shows a device stabilizing a fractured bone comprising an open resilient bracelet that contacts the bone at several support points.

U.S. Pat. No. 5,607,430 teaches a bone stabilization implant comprising a bone plate portion having a length with a center line, a bone contact surface and an outer surface. The bone plate portion includes a plurality of openings to receive bone screws extending through the outer surface and the bone contact surface located the length thereof. The outer surface includes a plurality of integral bosses positioned along the length thereof. Each of the bosses has crimping surfaces and a pair of holes therethrough to receive cable. Each of the bosses is deformable to clamp cable received in the pair of holes when the crimping surfaces are crimped. The integration of the bosses with the underlying bone plate portion better facilitates handling of the implant during implantation procedures and substantially reduces the breakage and wear problems associated with prior art bone stabilization implants.

U.S. Pat. No. 5,810,824 shows a surgical fastener assembly for use in bone fracture fixation comprises an adapter, a surgical cable, a compression plate and a crimp device. The adapter includes a head portion having a bore extending therethrough to receive the surgical cable and a protrusion extending from an underside of the head portion for insertion into a recess in the compression plate. The crimp device is deformable upon the cable to hold the cable in place around bone portions.

U.S. Pat. No. 5,993,452 relates to cerclage system for securing a fractured or weakened bone within a patient's body comprising a band to encircle the bone. A jacket supports the band in a spaced relationship from the bone.

U.S. Pat. No. 6,338,734 discloses an implant for use in fixing a resected portion of a bone to a proximal portion of the bone comprising an anatomically shaped plate member having a generally straight first portion and a second portion. The first portion has a bone receiving groove and at least one lower wire receiving groove. The bone receiving groove is adapted to receive a generally straight portion of the proximal portion of the bone. The second portion is coupled to the first portion at a first end and terminates at a distal end in at least one inwardly directed hook-shaped member. The second portion is curved to generally match the contour of an end portion of the bone. The hook-shaped member is adapted to abut the resected portion of the bone to prevent the resected portion from migrating from the straight portion of the proximal portion of the bone.

U.S. Pat. No. 7,250,054 shows a device for clamping and reclamping an orthopedic surgical cable used in conjunction with an orthopedic implant device, a bone, and/or bone implant or structure. Systems and methods provide a clamping body, a clamping mechanism, and a force application member that cooperate to allow tensioning and re-tensioning of a surgical cable.

U.S. Pat. No. 7,229,444 describes a bone fixation system comprising at least one cerclage cable and an elongate bone plate. The bone plate has a bone contacting surface, first and second spaced side portions and an outer surface opposite said bone contacting surface. The elongate plate includes a plurality of apertures surrounded by the side portions. The apertures extend from the outer surface to the bone contacting surface and are spaced apart to form bridge portions between adjacent apertures. Each of the bridge portions including at least two pairs of bores extending between the first and second side portions for receiving cerclage cable. Each of the first and second side portions forming the sides of at least one of the apertures also has a cerclage wire bore extending therethrough.

U.S. Pat. No. 7,255,701 teaches a method for clamping and reclamping an orthopedic surgical cable used in conjunction with an orthopedic implant device, a bone, and/or bone implant or structure. The sequence for the method comprises providing a surgical cable clamp and an orthopedic surgical cable, mounting a portion of the surgical cable to the surgical cable clamp, applying a force to the portion of the surgical cable so that the surgical cable is restrained or secured relative to the surgical cable clamp with a first tension in the surgical cable, releasing the force on the extended portion of the surgical cable so that the surgical cable can be repositioned relative to the surgical cable clamp and applying another force to the extended portion of the surgical cable so that the surgical cable is again restrained or secured relative to the surgical cable clamp.

U.S. 2003/0225410 shows a filament retaining washer mounted on the shank of a bone screw deployed in a bone to retain the filament to the bone comprising a structure having a screw hole extending therethrough to receive a bone screw to be deployed in the bone to secure the structure to the bone.

U.S. 2006/0235401 disclosed is a method and apparatus for repairing the femur comprising a connector having a claw-like member to engage the greater trochanter. Along the length of the connector are a plurality of cable apertures and cable screws to receive and engage the cables that loop around the femur. Along the inferior end of the connector are bone screw slots and bone screws engaging the connector with the femur.

Additional examples of the prior art are found in: U.S. Pat. No. 3,025,853; U.S. Pat. No. 4,795,473; U.S. Pat. No. 5,015,248; U.S. Pat. No. 5,041,114; U.S. Pat. No. 5,190,545; U.S. Pat. No. 5,397,328; U.S. Pat. No. 5,665,089; U.S. Pat. No. 5,702,399; U.S. Pat. No. 5,741,259; U.S. Pat. No. 5,973,223; U.S. Pat. No. 6,261,290; U.S. Pat. No. 6,280,446; U.S. Pat. No. 6,443,955; U.S. Pat. No. 6,461,360; U.S. Pat. No. 6,682,533; U.S. Pat. No. 6,960,213; U.S. Pat. No. 7,207,993; U.S. Pat. No. 7,341,588; U.S. Pat. No. 7,833,254; U.S. 2004/0087955; and U.S. 2006/0129151.

### SUMMARY OF THE INVENTION

The present invention relates to a fixation clamp to stabilize a bone fracture in an elongated tubular bone comprising a pair of arcuate clamp members pivotally coupled or hinged to each other by a hinge or pivot pin.

Each arcuate clamp member includes a concave inner surface having at least one spike-like element or protrusion extending outwardly from each concave inner surface to engage the surface of the bone to secure the fixation device in place. In addition, at least one pressure relief recess or indentation is formed in the concave inner surface of each clamp member to preserve the blood flow in the patient's limb when the fixation clamp is secured in position.

The fixation clamp further includes an adjustment device to tighten the arcuate clamp members around the bone forcing the spike-like elements or protrusions into the surface of the bone.

In addition, the fixation clamp may comprise a part of a fixation assembly. Specifically, the fixation assembly includes a bone plate having a plurality of internally threaded apertures formed along the length thereof to receive a corresponding plurality of externally threaded screws or fasteners to secure the bone plate to the patient's bone. At least one fixation clamp is then placed around the bone and over the bone plate aligning the internally threaded apertures of the fixation clamp with a corresponding internally threaded aperture of the bone plate to insert a corresponding anchoring screw or fastener therethrough and into the bone.

The invention accordingly comprises the features of construction, combination of elements, and arrangement of parts which will be exemplified in the construction hereinafter set forth, and the scope of the invention will be indicated in the claims.

### BRIEF DESCRIPTION OF THE DRAWINGS

For a fuller understanding of the nature and object of the invention, reference should be had to the following detailed description taken in connection with the accompanying drawings in which:

FIG. 1 is a side view of the fixation clamp of the present invention secured in operative position on a fractured bone.

FIG. 2 is an exploded top view of the fixation clamp of the present invention.

FIG. 3 is a partial side view of the fixation clamp of the present invention.

FIG. 4 is a side view of a fractured bone with a plurality of fixation clamps of the present invention in combination with an elongated plate secured in operative position on the fractured bone.

FIG. 5 is a side view of a fractured bone with a plurality of fixation clamps embodying the present invention in combination with an elongated plate secured in operative position on a fractured bone.

FIG. 6 is a top view of an alternate embodiment of the fixation clamp of the present invention.

FIG. 7 is a top view of an alternate embodiment of the fixation clamp shown in FIG. 6 operatively affixed to the bone of a patient.

Similar reference characters refer to similar parts throughout the several views of the drawings.

### DETAILED DESCRIPTION OF THE PREFERRED EMBODIMENT

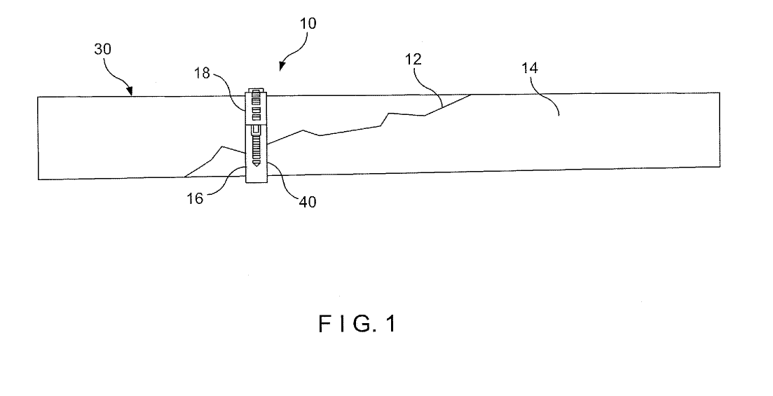

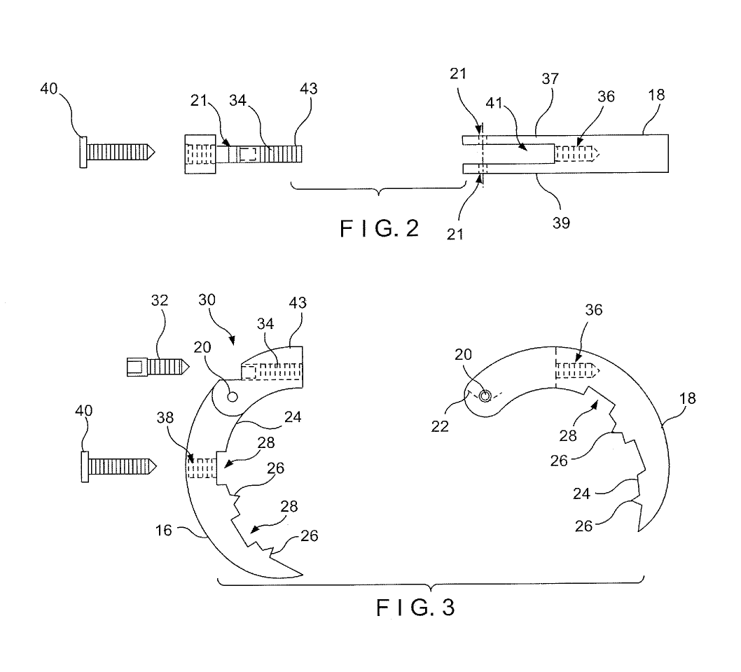

As shown in FIGS. 1 and 2, the present invention relates to a fixation clamp generally indicated as 10 to stabilize a spiral bone fracture 12 in an elongated tubular bone 14.

The fixation clamp 10 comprises a first arcuate clamp member 16 and a second arcuate clamp member 18 (a pair of clamp members) pivotally coupled or hinged to each other by a hinge or pivot pin 20 operatively disposed in channels or holes 21 formed in each of the clamp members 16 and 18. A bias or spring 22 is disposed adjacent the hinge or pivot pin 20 to normally bias the first arcuate clamp member 16 and the second clamp member 18 in an open position relative to each other.

Each arcuate clamp member 16 and 18 includes a concave inner surface 24 having at least one spike-like element or protrusion 26 extending outwardly from each concave inner surface 24 to engage the surface of the bone 14 .to secure the fixation clamp 10 in place. In addition, a plurality of pressure relief recesses or indentations each indicated as 28 is formed in the concave inner surface 24 of each clamp member 16 and 18 to preserve the blood flow in the patient's limb when the fixation clamp 10 is secured in position.

As shown in FIGS. 2 and 3, the fixation clamp 10 further includes an adjustment device generally indicated as 30 to tighten the arcuate clamp members 16 and 18 around the bone 14 forcing the spike-like elements or protrusions 26 into the surface of the bone 14. The adjustment device 30 comprises an externally threaded member 32 configured to be threaded through an internally threaded channel or portion 34 formed in the first arcuate clamp member 16 adjacent the hinge or pivot pin 20 and into an internally threaded portion or recess 36 formed in the second arcuate clamp member 18 adjacent the hinge or pivot pin 20 to tighten and secure the fixation clamp 10 in place around the bone 14.

The second arcuate clamp member 18 includes an upper plate or flange 37 and a lower plate or flange 39 disposed in spaced relationship relative to each other to cooperatively form a slot or opening 41 therebetween to receive an extension or tongue 43 including the internally threaded channel or portion 34 formed on the first arcuate clamp member 16. The fixation clamp 10 may further include at least one internally threaded aperture or channel 38 formed through one or both arcuate clamp members 16 and 18 to receive an externally threaded anchoring screw or fastener 40 screwed into the bone 14 to anchor the fixation clamp 10 in place.

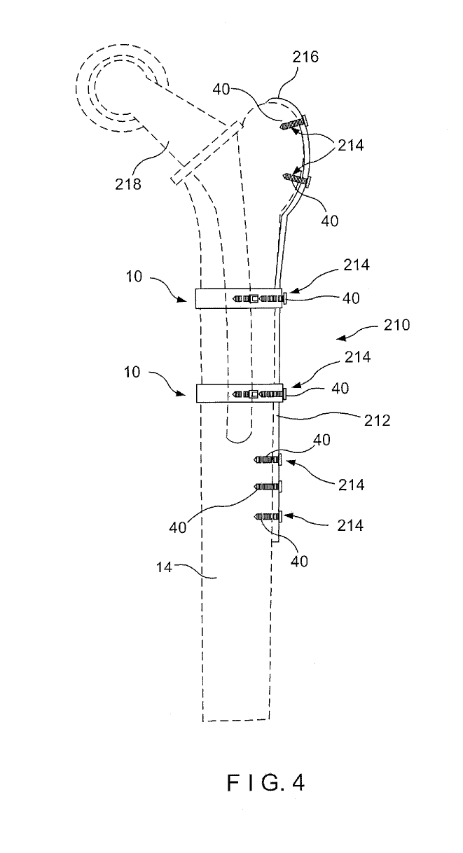

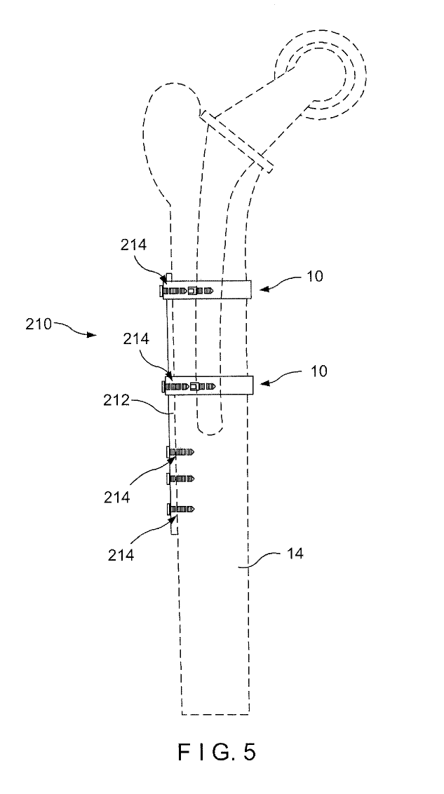

FIGS. 4 and 5 show the fixation clamp 10 in use as part of a fixation assembly. Specifically, in addition to the fixation clamp 10, the fixation assembly includes a bone plate generally indicated as 210 comprising an elongated plate member 212 having a plurality of internally threaded apertures each indicated as 214 formed along the length thereof and a trochanteric grip portion 216 formed on the upper end portion of the elongated plate member 212 having at least one internally threaded aperture 218 to receive a corresponding plurality of externally threaded screws or fasteners 40 to secure the bone plate 210 to the patient's bone 14 including the femur adjacent the hip prosthesis 218. At least one fixation clamp 10 is then placed around the bone 14 and over the elongated plate member 212 of the bone plate 210 aligning the internally threaded aperture 38 of one of the pair of clamp members 16 and 18 with corresponding internally threaded aperture 214 to insert a corresponding anchoring screw or fastener 40 therethrough and into the bone 14.

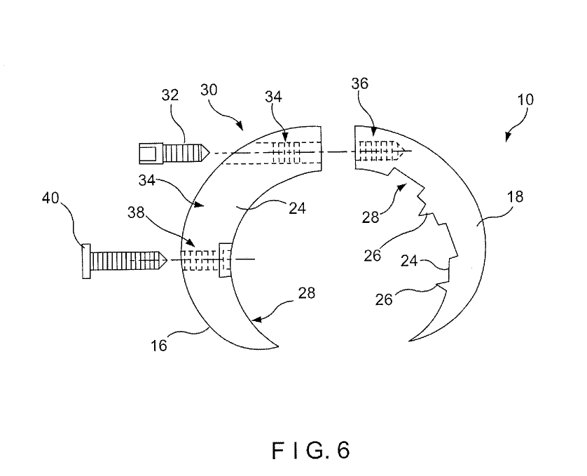

FIG. 6 shows an alternate embodiment of the present invention. Specifically, the fixation clamp 10 comprises a first arcuate clamp member 16 and a second arcuate clamp member 18 (a pair of separate clamp members).

Each arcuate clamp member 16 and 18 includes a concave inner surface 24 at least one of which has at least one spike-like element or protrusion 26 extending outwardly from the concave inner surface 24 to engage the surface of the bone 14 to secure the fixation clamp 10 in place. In addition, at least one pressure relief recess or indentation 28 is formed in the concave inner surface 24 of at least one of the clamp members to preserve the blood flow in the patient's limb when the fixation clamp 10 is secured in position.

The fixation clamp 10 further includes an adjustment device generally indicated as 30 to tighten the arcuate clamp members 16 and 18 together around the bone 14 forcing the spike-like element or protrusion 26 into the surface of the bone 14. The adjustment device 30 comprises an externally threaded member 32 configured to be threaded through an internally threaded portion or channel 34 formed in the first arcuate clamp member 16 and into an internally threaded portion or recess 36 formed in the second arcuate clamp member 18 to tighten and secure the fixation clamp 10 in place around the bone 14.

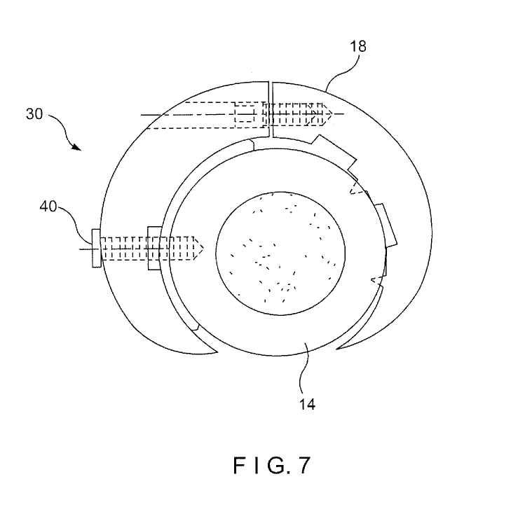

As best shown in FIG. 7, the first arcuate clamp member 16 and the second arcuate clamp member 18 do not fully encircle the bone 14 leaving a space between the free or distal ends to insure continued circulation.

It will thus be seen that the objects set forth above, among those made apparent from the preceding description are efficiently attained and since certain changes may be made in the above construction without departing from the scope of the invention, it is intended that all matter contained in the above description or shown in the accompanying drawing shall be interpreted as illustrative and not in a limiting sense.

It is also to be understood that the following claims are intended to cover all of the generic and specific features of the invention herein described, and all statements of the scope of the invention which, as a matter of language, might be said to fall therebetween.

What is claimed is:

1. A fixation clamp to stabilize a bone fracture comprising a pair of arcuate clamp members each including a concave inner surface coupled to each other and an adjustment device to tighten said arcuate clamp members around the bone.

2. The fixation clamp of claim 1 wherein said pair of arcuate clamp members are pivotally coupled to each other by a pivot pin.

3. The fixation clamp of claim 2 further including a bias disposed adjacent said pivot pin to normally bias said arcuate clamp members apart relative to each other.

4. The fixation clamp of claim 2 wherein said adjustment device comprises an externally threaded member configured to be threaded through an internally threaded portion formed in one of said arcuate clamp members adjacent said pivot pin and into an internally threaded portion formed in the other of said arcuate clamp members adjacent said pivot pin to tighten and secure said fixation clamp in place around the bone.

5. The fixation clamp of claim 4 wherein each said arcuate clamp member includes a concave inner surface having at least one protrusion extending outwardly from each concave inner surface to engage the surface of the bone to secure said fixation clamp in place.

6. The fixation clamp of claim 5 further including at least one pressure relief recess formed in said concave inner surface of at least one of said clamp members to preserve the blood flow in the patient's limb when said fixation clamp is secured in position.

7. The fixation clamp of claim 4 further including at least one pressure relief recess formed in the concave inner surface of each clamp member to preserve the blood flow in the patient's limb when said fixation clamp is secured in position.

8. The fixation clamp of claim 7 further including at least one internally threaded aperture formed through one or both of said arcuate clamp members to receive an externally threaded anchoring screw to anchor said fixation clamp in place.

9. The fixation clamp of claim 4 further including at least one internally threaded aperture formed through one or both of said arcuate clamp members to receive an externally threaded anchoring screw to anchor said fixation clamp in place

10. The fixation clamp of claim 2 further including at least one internally threaded aperture formed through one or both of said arcuate clamp members to receive an externally threaded anchoring screw to anchor said fixation clamp in place.

11. A fixation assembly to stabilize a bone fracture comprising a bone plate including at least one internally threaded aperture formed therethrough to receive an externally threaded anchor screw to fasten said bone plate to the bone and a fixation clamp comprising a pair of arcuate clamp members each including a concave inner surface coupled to each other and an adjustment device to tighten said arcuate clamp members around said bone plate and the bone.

12. The fixation clamp of claim 11 wherein said pair of arcuate clamp members are pivotally coupled to each other by a pivot pin.

13. The fixation clamp of claim 12 further including a bias disposed adjacent said pivot pin to normally bias said arcuate clamp members apart relative to each other.

14. The fixation clamp of claim 12 wherein said adjustment device comprises an externally threaded member configured to be threaded through an internally threaded portion formed in one of said arcuate clamp members adjacent said pivot pin and into an internally threaded portion formed in the other of said arcuate clamp members adjacent said pivot pin to tighten and secure said fixation clamp in place around the bone.

15. The fixation clamp of claim 14 wherein each said arcuate clamp member includes a concave inner surface having at least one protrusion extending outwardly from each concave inner surface to engage the surface of the bone to secure said fixation clamp in place.

16. The fixation clamp of claim 15 further including at least one pressure relief recess formed in said concave inner surface of at least one of said clamp members to preserve the blood flow in the patient's limb when said fixation clamp is secured in position.

17. The fixation clamp of claim 14 further including at least one pressure relief recess formed in the concave inner surface of each clamp member to preserve the blood flow in the patient's limb when said fixation clamp is secured in position.

18. The fixation clamp of claim 17 further including at least one internally threaded aperture formed through one or both of said arcuate clamp members to receive an externally threaded anchoring screw to anchor said fixation clamp in place.

19. The fixation clamp of claim 14 further including at least one internally threaded aperture formed through one or both of said arcuate clamp members to receive an externally threaded anchoring screw to anchor said fixation clamp in place.

20. The fixation clamp of claim 12 further including at least one internally threaded aperture formed through one or both arcuate clamp members to receive an externally threaded anchoring screw to anchor said fixation clamp in place.

21. The fixation clamp of claim 11 wherein one of said clamp members includes an internally threaded aperture formed therethrough to be aligned with said internally threaded aperture formed in said bone plate such that each aperture receives the same externally threaded anchoring screw therethrough to anchor said fixation clamp and said bone plate in place.

22. The fixation clamp of claim 1 wherein said fixation clamp does not fully encircle the bone when operatively affixed to a patient.

23. The fixation clamp of claim 11 wherein said fixation clamp does not fully encircle the bone when operatively affixed to a patient.
